(19) United States
(12) Patent Application Publication
(10) Pub. No.: US 2017/0119234 A1
(43) Pub. Date: May 4, 2017

(54) ENDOSCOPE CAP WITH SEPARABLE ARMS

(71) Applicant: Cook Medical Technologies LLC, Bloomington, IN (US)

(72) Inventor: David Petroskey, Lorain, OH (US)

(73) Assignee: Cook Medical Technologies LLC, Bloomington, IN (US)

(21) Appl. No.: 15/289,385

(22) Filed: Oct. 10, 2016

Related U.S. Application Data
(60) Provisional application No. 62/241,423, filed on Oct. 14, 2015.

(51) Int. Cl. A61B 1/00

(52) U.S. Cl. CPC A61B 1/00089; A61B 1/00096; A61B 1/00101; A61B 1/00137

(57) ABSTRACT

The present embodiments provide a cap for attachment to a distal end of an endoscope. The cap may comprise a proximal portion comprising a tubular body with a lumen extending therethrough, wherein the proximal end is configured for securement to the distal end of the endoscope, and a distal portion having a first arm extending to a first distal end, the first arm being movable such that a first end of the first arm is moveable laterally with respect to a longitudinal axis defined by the tubular body.

### CROSS-REFERENCE TO RELATED APPLICATIONS

This application is a non-provisional application which claims priority to U.S. provisional application Ser. No. 62/241,423, filed Oct. 14, 2015, which is incorporated by reference herein in its entirety.

### BACKGROUND

Endoscopic devices and procedures may be used to diagnose, monitor and treat various conditions by close examination of the internal organs. By way of background, a conventional endoscope generally is an instrument having a device for visualizing the interior of an internal region of a body and a lumen for inserting one or more treatment devices therethrough. A wide range of applications have been developed for the general field of endoscopes including by way of example the following: arthroscope, angioscope, bronchoscope, choledochoscope, colonoscope, cytoscope, duodenoscope, enteroscope, esophagogastro-duodenoscope (gastroscope), laparoscope, laryngoscope, nasopharyngo-neproscope, sigmoidoscope, thoracoscope, and utererscope (individually and collectively, “endoscope”).

An endoscope may be useful to treat one of the several disorders of the gastrointestinal tract, e.g., gastrointestinal inflammation, gastrointestinal cancer, gastrointestinal infection, gastrointestinal motility dysfunction, or lesions, wounds or contusions of tissue of a portion of the gastrointestinal tract that can cause gastrointestinal lesions. There is a wide variety of medical procedures that require removal or dissection of the mucosal or submucosal layers of gastrointestinal tract wall to treat these disorders.

To treat motility disorders from within the esophagus, a procedure called per-oral endoscopic myotomy (“POEM”) may be used. In this procedure, a tunnel is generally formed beneath the mucosal and submucosal layers such that a delivery device (e.g., an endoscope with a working channel) may access the underlying layers of muscle tissue. The top muscle layer may then be cut in the axial direction, which may weaken tightness in the esophagus to treat the motility disorder.

Currently, a high level of skill and attention is required access the muscle layer. The target area where it is desirable to perform the cut is near vital arteries and is generally at a location in a position of the body that is difficult to reach, and therefore it is difficult for a medical professional to precisely operate at the target location. Further, because several layers of tissue, such as mucosal and submucosal layers, may cover the target muscle area, viewing devices may be blocked at the target site, thereby severely limiting the visual feedback.

Accordingly, it would be desirable to provide an improved device located at the end of an endoscope which may assist in tunneling through tissue layers, may facilitate uncovering of a target area for performance of a medical procedure, and may improve the visual feedback provided to a medical professional.

### BRIEF SUMMARY

The present embodiments provide a cap for attachment to a distal end of an endoscope. The cap may comprise a proximal portion comprising a tubular body with a lumen extending therethrough, wherein the proximal end is configured for securement to the distal end of the endoscope. The cap may further comprise a distal portion having a first arm extending to a first distal end, the first arm being movable such that a first end of the first arm is moveable laterally with respect to a longitudinal axis defined by the tubular body.

In some embodiments, the cap has activator in contact with a first surface of the first arm, the activator being moveable to effect the movement of the first distal end of the first arm. The distal portion of the cap may further comprise a second arm, the second arm being movable such that a second distal end of the second arm is moveable laterally with respect to the longitudinal axis defined by the tubular body. The cap may have an activator disposed at least partially between the first arm and the second arm, wherein a contact portion of the activator is movable in the proximal direction to laterally spread the first distal end of the first arm from the second distal end of the second arm.

The activator can be connected to a cable, the cable being configured to provide a force on the activator in a proximal direction. The first arm may comprise a hinge, the hinge being secured to the proximal portion of the cap and allowing lateral movement of the first distal end of the first arm. In some embodiments, the cap further comprises a spring circumnavigating the distal portion of the cap and configured to provide an inward lateral force on the first arm towards the longitudinal axis defined by the tubular body. The contact portion of the activator may be configured to slideably contact the first surface of the first arm.

### BRIEF DESCRIPTION OF THE DRAWINGS

FIG. 1 is a front view of a cap for attachment to a distal end of an endoscope in an open configuration in accordance with an embodiment of the present invention.

FIG. 2 is a front cutout view of a cap for attachment to a distal end of an endoscope with a first arm in accordance with an embodiment of the present invention.

FIG. 3 is a front view of a cap attached to the distal end of an endoscope and in an open configuration in an esophagus of a patient in accordance with an embodiment of the present invention.

FIG. 4 is a side, rotated view of a cap for attachment to a distal end of an endoscope in accordance with an embodiment of the present invention.

FIG. 5 is a front view of a cap for attachment to a distal end of an endoscope in a closed configuration in accordance with an embodiment of the present invention.

FIG. 6 is a front view of a cap for attachment to a distal end of an endoscope in an open configuration in accordance with an embodiment of the present invention.

### DETAILED DESCRIPTION OF THE DRAWINGS

The invention is described with reference to the drawings in which like elements are referred to by like numerals. The relationship and functioning of the various elements of this invention are better understood by the following detailed description. However, the embodiments of this invention are not limited to the embodiments illustrated in the drawings. It should be understood that the drawings are not to scale, and in certain instances details have been omitted which are not necessary for an understanding of the present invention, such as conventional fabrication and assembly.

As used in the specification, the terms proximal and distal should be understood as being in the terms of a physician delivering cap to a patient. Hence the term “distal” means the portion of the cap that is farthest from the physician and the term “proximal” means the portion of the cap that is nearest to the physician.

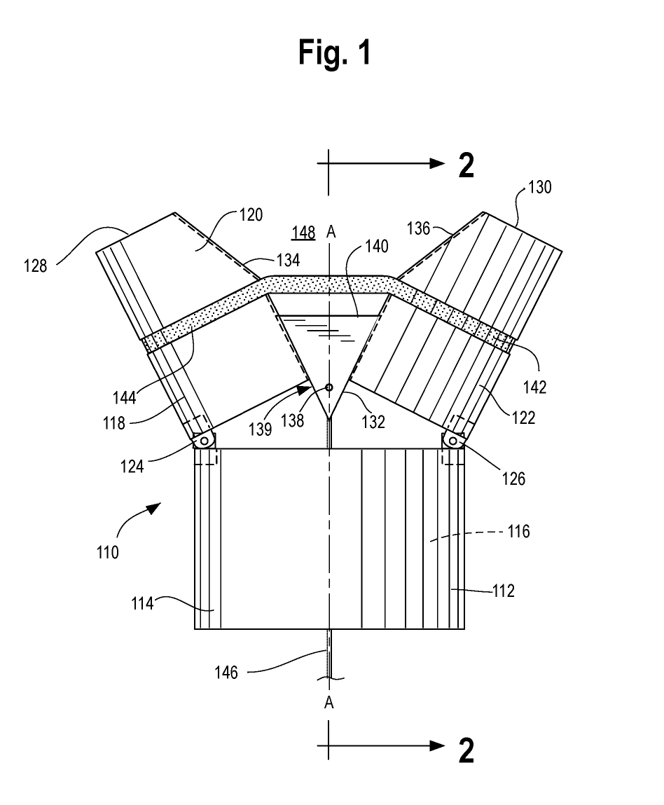

FIG. 1 illustrates one embodiment of a cap 110. As shown in FIG. 1, the cap 110 may include a tubular body 114 at a proximal portion 112 of the cap 110, the tubular body 114 having a lumen 116 formed therein. The proximal portion 112 may be secured to a medical device, such as an endoscope 102 (see FIG. 3), and may be configured such that the tubular body 114 fits over the distal end of the endoscope. In some embodiments, additional suitable devices may be included and configured to facilitate this described securement, such as an elastomeric portion as described by U.S. Patent Application Publication 2014/0100570 to McLawhorn, which is incorporated by reference herein in its entirety. Further, the cap 110 may be a multipart cap being universally connectable to many different diameter endoscopes, as described by U.S. Patent Application Publication 2013/0046138, also to McLawhorn, which is incorporated by reference herein in its entirety.

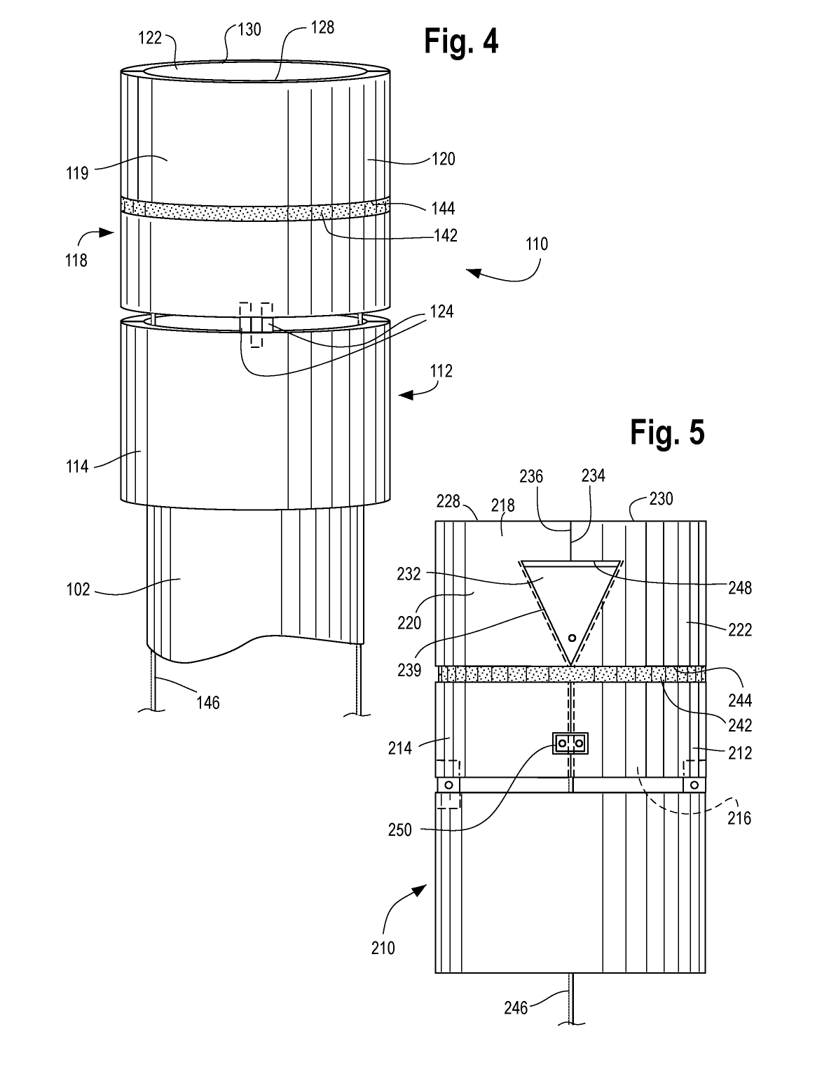

Referring to the embodiment of FIG. 1, the cap 110 comprises a distal portion 118 having a first arm 120 and a second arm 122. The first arm 120 and the second arm 122 may be connected to the proximal portion 112 through a first hinge 124 and second hinge 126, respectively. The distal portion 118 (and potentially at least a portion of the proximal portion 112) may extend distally from the endoscope 102 (see FIG. 4). In other embodiments, only one arm or more than two arms may be provided. The first arm 120 may extend to a first distal end 128, and the second arm 122 may have a second distal end 130. The first arm 120 may be moveable (e.g., may be pivotable at first hinge 124) such that the first distal end 128 is movable laterally (i.e., substantially radially towards and away an axis) with respect to a longitudinal axis A defined by the tubular body 114. Similarly, the second arm 122 may be moveable (e.g., may be pivotable at second hinge 126) such that the second distal end 130 is moveable laterally with respect to the longitudinal axis A of the tubular body 114. In other words, the arms 120 and 122 may comprise an open state (depicted in FIG. 1) and a closed state (depicted in FIGS. 4 and 5), wherein in the open state, the distal ends 128 and 130 are positioned farther away from the longitudinal axis A defined by the tubular body 114 than they are in the closed state. One or more selectable intermediate states may also be possible. In some embodiments, the arms 120, 122 may be cooperatively laterally movable to the open state. Further, the first arm 120 or the second arm 122 may be positioned a different distance away from the longitudinal axis A than the other of the first arm 120 and the second arm 122 in the open state (or any other state). In the closed state depicted by FIG. 4, the cap 110 may be substantially tubular with lumen 116 extending through the proximal portion 112 and the distal portion 118. As shown, the arms 120, 122 may be sized and shaped in a curved configuration so that surfaces 134, 136 of the arms 120, 122 meet in the closed state and form a second tubular body 119. In some embodiments, the distal portion 118 in the closed state may have an outer diameter substantially equal to an outer diameter of the proximal portion 112. In other embodiments, the outer diameter of the distal portion 118 may be smaller than or greater than the outer diameter of the proximal portion 112.

In some embodiments, certain portions of the cap 110, including, for example, the tubular body 114 and the arms 120 and 122, may be made primarily of a substantially transparent or translucent polymer such as polytetrafluorothylene (PTFE). Additional possible materials include, but are not limited to the following, polyethylene ether ketone (PEEK), fluorinated ethylene propylene (FEP), perfluoroalkoxy polymer resin (PFA), polyamide, polyurethane, high density or low density polyethylene, and nylon. In some embodiments, the cap may be formed from a lubricious material such as PTFE and the like for easy slidability within the patient's lumen for delivery to the treatment site. The cap or a portion thereof may also be coated or impregnated with other compounds and materials to achieve the desired properties. Exemplary coatings or additives include, but are not limited to, parylene, glass fillers, silicone hydrogel polymers and hydrophilic coatings.

As shown in FIG. 1, the cap 110 may include an activator 132 with at least one contact portion 139. The activator 132 may be shaped as a wedge, as shown. The contact portion 139 may be, for example, a contact surface. The activator 132 may be operable to separate the arms 120 and 122 and/or to control the orientation of the arms 120 and 122, as shown in FIG. 1. When the arms 120 and 122 are separated, a space 148 may be provided. In some embodiments, at least a portion of this space 148 (e.g., between surfaces 134 and 136) may be covered by, for example, a cloth or another material which is preferably (though not necessarily) transparent. In other embodiments (for example, where only one arm is provided/movable), the activator 132 may separate a single arm from, for example, a stationary portion of the distal portion 118. The activator 132 may be moveable in the distal and proximal directions with respect to at least one of the arms 120 and 122, and may include a contact portion 139 is slideable relative to a surface of an arm, such as a first surface 134 of the first arm 120 and/or a second surface 136 of the second arm 122. In the depicted embodiment, when the activator 132 moves proximally, the contact portion 139 provides a separating force against the surfaces 134 and 136, thereby moving the distal ends 128 and 130 laterally away from the longitudinal axis A. The activator 132 may be generally triangular shaped, as depicted in FIG. 1, and may comprise a narrow portion 138 facing proximally and a wide portion 140 facing distally. The shape and size of the activator 132 may determine the range of the movement of the arms 120 and 122.

More than one activator may be provided. For example, in some embodiments, an activator may be provided at both locations where the first arm 120 and second arm 122 split (e.g., the front and back of the cap 110 from the perspective of FIG. 1). In some embodiments, a single activator may pass across the lumen 116 to act at both locations where the first arm 120 and second arm 122 split, though in other embodiments, the activator 132 is sized to avoid substantially extending from surfaces 134 and 136, which may prevent substantially interfering with working space of the device. In the depicted embodiment of FIG. 1, the activator 132 is be symmetric so that the first arm 120 and the second arm 122 move equal distances away from and toward the longitudinal axis A when the activator 132 is move proximally and distally, respectively. In other embodiments, the activator 132 may be asymmetric so that the first arm 120 and the second arm 122 move different distances away from and toward the longitudinal axis A when the activator 132 is move proximally and distally, respectively. Further, the cap may include markings to provide an indication of how far the arms 120, 122 are opened. In some instances, there may be different optimal distances for movement from or toward the longitudinal axis A, and therefore providing markings or other forms of indicators may be advantageous to help an operator know the location of the arms 120, 122 and provide necessary adjustments to achieve optimal positioning. These markings or other types of indicators may be provided on the cap 110 (such as on the lumen 116 or the cable 146) or may be provided on an attached device (such as the endoscope). The markings may be associated with markings on the cable 146.

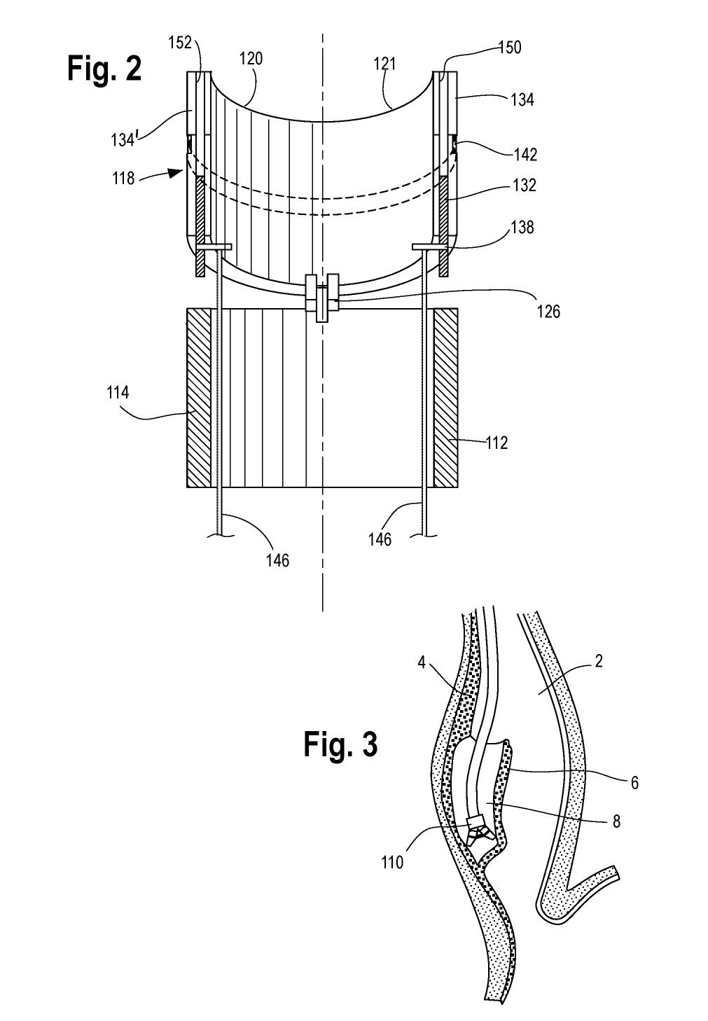

In some embodiments, and referring to FIG. 1 and FIG. 2, at least a portion of the activator (e.g., a contact portion 139, shown in FIG. 1) may be configured to fit within a groove 150 located at the surface 134 of the first arm 120 (shown in FIG. 2). The second arm 122 may comprise a similar groove. In some embodiments, a portion of the activator 132, such as contact portion 139, is slideable within the groove 150. The groove 150 preferably retains the activator 132 such that the activator does not separate from the arm 120, and may provide securement of the activator 132 to other elements of the cap 110. The contact between the contact portion 139 of the activator 132 and the groove 150 may be lubricated in some embodiments. In embodiments comprising two activators, a second groove 152 may be included on a depicted surface 134′ of the arm 120 opposite of surface 134. Similarly, the second arm 122 may comprise two grooves on two surfaces or edges. Alternatively, or in addition, a groove could be located on an inner surface 121 of the first arm 120. In other embodiments, the activator 132 may be connected to one or more arms without the use of a groove and/or by utilizing another suitable connection device or method. Further, there may be a spring housed in the groove 150 to provide a bias on the contact portion 139 of the activator 132.

As depicted in FIG. 1, the distal portion 118 may comprise a spring 142 surrounding an outer perimeter of the distal portion 118. The spring 142 may be configured to provide a constricting force on the arms 120 and 122, which may bias the arms 120 and 122 toward a closed position (shown in FIG. 4). The spring 142 may be seated in a groove 144 located on the outer surfaces of arms 120 and 122, as shown. In some embodiments, the spring 142 may be covered to prevent snagging or potential damage to tissue adjacent to the spring 142. Alternatively, the spring 142 may be contained within a cavity formed by the arms 120 and 122, or may be located adjacent to the inner surface of the arms 120 and 122. It may not be necessary for the spring 142 to completely surround the distal portion 118 in all embodiments.

As shown in FIG. 1, the activator 132 may be attached to a cable 146, which may be controllable by a medical professional operating the cap 110. In some embodiments, the cable 146 may extend to the proximal end of the endoscope 102 (see FIG. 4). The cable 146 may be configured to pull the activator 132 proximally or otherwise provide a force on the activator 132 such that the activator 132 moves proximally to open the arms 120 and 122. In some embodiments, the cable 146 may be sufficiently ridged as to be capable of also providing a force in the distal direction on the activator 132, which may provide an operator with the ability to directly control distal movement of the activator 132 to thereby close the arms 120 and 122. The cable 146 may be located at least partially within the lumen 116, and may extend proximally of the tubular body 114 through the lumen of the endoscope. Other embodiments may provide a cable located on the outside of the tubular body 114.

FIG. 3 depicts the cap 110 in the open configuration within a submucosal tunnel 8 in an esophagus 2. The submucosal tunnel 8 may be formed between the mucosal and submucosal layers 6 and underlying muscle tissue 4. The arms (see FIG. 1) of cap 110 are shown as providing a working and visual space 148 distally of the endoscope 102, which may, as described herein, increase the ability of an operator to visualize within the space 148 and adjacent tissue, as well as provide additional space for the operation of a medical procedure (such as a POEM procedure) at space 148.

FIG. 4 depicts an embodiment of the cap 110 in a closed configuration. The cap 110 comprises the proximal portion 112 with the tubular body 114 connected to an endoscope 102. The distal portion 118 comprises the first arm 120 and the second arm 122 depicted in a closed configuration. In the closed configuration, the first distal end 128 of the first arm 120 and the second distal end 130 of the second arm 122 may contact or otherwise be adjacent to one another. Further, in the closed configuration, the distal portion 118 may form a substantially tubular body 119 with approximately the same diameter as the tubular body 114 of the proximal portion 112. Spring 142, which is seated in a groove 144, circumnavigates the distal portion 118, thereby biasing the arms 120 and 122 to remain in the closed configuration. The arms 120 and 122 may be, as described above, attached to the proximal portion 112 at hinges (e.g., the first arm 120 is attached at hinges 124). In FIG. 4, the activator 132 is hidden, though it should be noted that the cap 110 may be made of a transparent or translucent material, as described above.

Referring to FIG. 5, in some embodiments, the cap 210 may comprise a distal portion 218 with a first arm 220 and a second arm 222 forming a cavity 248, which is shaped to surround an activator 232. The cavity 248 may house the activator 232 when, for example, the first arm 220 and the second arm 222 are in a closed orientation (as depicted). This embodiment may be advantageous, as the activator 232 does not need to be positioned distally of the distal portion 218 when the arms 220 and 222 are in the described closed orientation. The cavity 248 may be located at any position along depicted separable surfaces 234 and/or 236, such as approximately in the longitudinal middle of the distal portion 218 (as depicted), though providing the cavity 248 adjacent to or near a first distal end 228 of the first arm 220 and/or a second distal end 230 of the second arm 222 may maximize the range of movement of the first and second arms 220 and 222.

As previously described, a spring 242, which rests in a groove 244, may provide a tendency for the arms 220 and 222 to close when the activator 232 is not forcing them towards an open orientation. In some embodiments, the activator 232 is sufficiently movable (e.g., a contact surface 239 is sufficiently slidable along surfaces 234 and 234) such that, when force is released from a cable 246, the spring 242 may provide a sufficient constricting force, which can be mechanically translated into a distal force contact surface 239 from the surfaces 234 and 236, to move the activator 232 upwards into cavity 248, thereby closing the arms 220 and 222. In other embodiments, the activator 232 may be forced upward a rigid cable 246 (as described above) or by another suitable device or method configured to move the activator 232 such that a closed and/or intermediate configuration can be achieved.

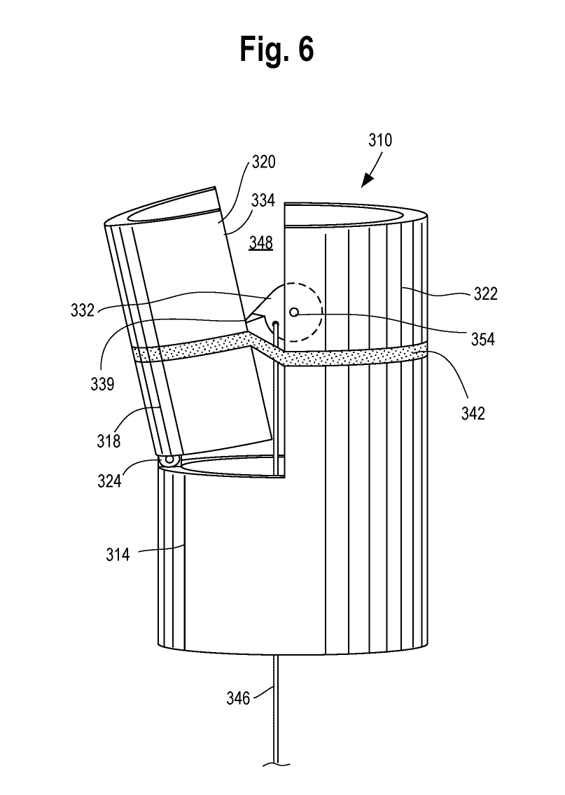

While the activator can operate as a wedge, as described above, it can alternatively and/or additionally be any other suitable device. As an illustration, in an alternative embodiment depicted in FIG. 6, a cap 310, shown in an open configuration, may comprise a rotatable activator 332. The activator 332 could be attached anywhere on the inside or outside of the cap 310. The rotatable activator 332 may be configured to separate a first arm 320 from a second arm 322 located on a distal portion 318 of the cap 310. As shown, the second arm 322 is integral to the proximal portion 314, and the first arm 320 is connected to the proximal portion 314 by way of a hinge 324. Alternatively, the second arm 322 could also be secured to the proximal portion 314 in a movable manner. The activator 332 is rotatably connected to the first arm 320 at a pivot point 354. A contact portion 339 of the activator 332 may be configured to contact a surface 334 of the first arm 320 to thereby effect the surface of the first arm 320.

Movement (e.g., rotation) of the activator 332, which may be influenced by a cable 346 controlled by an operator, can thereby influence the movement of the first arm 320 between a closed and the depicted open configuration. As depicted, the first arm 320 is in an open configuration and provides a space 348. The contact portion 339 may be enlarged, or may extend radially, such that it can contact the surface 334 (and in some embodiments, the contact portion 339 may extend radially and remain between the first arm 320 and the second arm 322 in intermediate states and the closed state). In some embodiments, the contact portion 339 may be slideably retained within an elongated groove or cavity of the surface 334 (not shown), and may therefore be capable of effecting the motion of the first arm 320 in two directions. As described above, a spring 342 may circumnavigate the distal portion 318 and may be configured to bias the distal portion 318 into the closed configuration. In addition (or alternatively), a rotation spring (e.g., a torsion spring, not shown) may be included and may create a bias for the activator to rotate into a position corresponding with the closed configuration.

The embodiments described herein have several advantageous characteristics. The separable arms described herein may separate such that they provide a force on two layers of adjacent tissue to facilitate separating those two layers during, for example, a POEM procedure, which may thereby facilitate tunneling under a tissue layer (e.g., the submucosa). Further, the separable arms may open and spread laterally, as described herein, to push tissue laterally and give exposure to underlying muscle, thereby allowing access by other instruments associated with the endoscope configured, for example, to perform a POEM procedure. This may additionally improve the view of a target area when using a device for visualizing the interior of an internal region of a body, particularly in embodiments where the arms and/or the other portions of the cap are made of a transparent or translucent material. The device further may be operated by a single medical professional, which may ease the process and reduce the need for assistance by a second user.

The figures and disclosure are intended to be illustrative and not exhaustive. This description will suggest many variations and alternatives to one of ordinary skill in the art. All such variations and alternatives are intended to be encompassed within the scope of the attached claims. Those familiar with the art may recognize other equivalents to the specific embodiments described herein which equivalents are also intended to be encompassed by the attached claims.

1. A cap for attachment to a distal end of an endoscope, the cap comprising:
a proximal portion comprising a tubular body with a lumen extending therethrough, wherein the proximal end is configured for securement to the distal end of the endoscope;
a distal portion having a first arm extending to a first distal end, the first arm being movable such that a first end of the first arm is moveable laterally with respect to a longitudinal axis defined by the tubular body.

2. The cap of claim 1, further comprising an activator in contact with a first surface of the first arm, the activator being moveable to effect the movement of the first distal end of the first arm.

3. The cap of claim 1, wherein the distal portion of the cap further comprises a second arm, the second arm being movable such that a second distal end of the second arm is moveable laterally with respect to the longitudinal axis defined by the tubular body.

4. The cap of claim 3, the cap further comprising an activator disposed at least partially between the first arm and the second arm, wherein a contact portion of the activator is movable in the proximal direction to laterally spread the first distal end of the first arm from the second distal end of the second arm.

5. The cap of claim 2, wherein the activator is connected to a cable, the cable being configured to provide a force on the activator in a proximal direction.

6. The cap of claim 1, wherein the first arm comprises a hinge, the hinge being secured to the proximal portion of the cap and allowing lateral movement of the first distal end of the first arm.

7. The cap of claim 1, wherein the cap further comprises a spring circumnavigating the distal portion of the cap and configured to provide an inward lateral force on the first arm towards the longitudinal axis defined by the tubular body.

8. The cap of claim 2, wherein the contact portion of the activator is configured to slideably contact the first surface of the first arm.

9. A cap for an endoscopic device, the cap comprising:
a proximal portion having a first tubular body configured to operably connect to a distal end of the endoscopic device; and
a distal portion having a first state and a second state,
wherein in the first state, a first arm comprises a first orientation wherein a first distal end of the first arm is a first distance from a longitudinal axis defined by the tubular body, and
wherein in the second state, the first arm comprises a second orientation wherein the first distal end of the first arm is a second distance from the longitudinal axis defined by the tubular body, the second distance being greater than the first distance.

10. The cap of claim 9, wherein in the second state, the distal portion defines a second tubular body adjacent to the first tubular body, wherein the lumen extends through the first and second tubular bodies.

11. The cap of claim 9, wherein the cap further comprises an activator with a contact portion being configured to contact a first surface of the first arm, the contact portion being movable at least partially in the proximal direction to effect lateral movement of the first arm away from the longitudinal axis.

12. The cap of claim 11, the cap further comprising a second arm with a second surface, wherein the activator is disposed at least partially between the first surface of the first arm and the second surface of the second arm, and wherein the activator is configured to spread the first arm from the second arm when moving in the proximal direction.

13. The cap of claim 11, the cap further comprising a cable connected to the activator and configured to provide a force on the activator in the proximal direction.

14. The cap of claim 9, wherein the first arm is connected to a first hinge secured to the proximal portion of the cap.

15. The cap of claim 9, the cap further comprising a spring circumnavigating the distal portion and configured to provide a lateral force on the first arm towards the longitudinal axis defined by the tubular body.

16. The cap of claim 9, wherein the contact portion of the activator is configured to slideably contact the first surface of the first arm.

17. A method for treating a patient, the method comprising:
deploying an endoscope with a cap to a target tissue area within a tunnel beneath underlying layers of patient tissue;
wherein the cap comprises
a proximal end with a tubular body configured for securement to the endoscope, and
a distal end with a first arm extending to a first distal end and connected to the proximal portion through a hinge;
moving the first arm such that the first arm pivots on the hinge, thereby moving the first distal end of the first arm laterally away from a longitudinal axis defined by the tubular body.

18. The method of claim 17, wherein the method further comprises forming a cut within a muscle layer at the target tissue area.

19. The method of claim 17, wherein the method further comprises moving a contact portion of an activator distally, the contact portion being in contact with a first surface of the first arm, and the contact portion being configured to provide a lateral force on the first arm to effect the motion of the first arm.

20. The method of claim 18, wherein the method further comprises moving the contact surface distally between at least a portion of the first arm and the second arm to thereby separate the two arms.
